(19) United States
(12) Patent Application Publication
(10) Pub. No.: US 2003/0087283 A1
(43) Pub. Date: May 8, 2003

(54) Use of fructose-1,6-bisphosphate aldolase for identifying new fungicidally active substances

(76) Inventors: Edda Koopmann, Koln (DE); Gabi Friedrich, Leverkusen (DE); Volker Gutsmann, Leichlingen (DE); Bernhard Grimmig, Langenfeld (DE); Karl-Heinz Kuck, Langenfeld (DE); Verena Vollenbroich, Mulheim (DE)

Correspondence Address: BAYER CORPORATION, PATENT DEPARTMENT, 100 BAYER ROAD, PITTSBURGH, PA, 15205, US

(21) Appl. No.: 10/230,033

(22) Filed: Aug. 28, 2002

(30) Foreign Application Priority Data
Aug. 30, 2001 (DE) 10142328.4

(51) Int. Cl. C12Q 1/68; C07H 21/04; C12Q 1/26; C12Q 1/18

(52) U.S. Cl. 435/6; 435/32; 435/25; 536/23.2

(57) ABSTRACT

The invention relates to nucleic acids which encode fungal polypeptides with the biological activity of fructose-1,6-bisphosphate aldolases, to the polypeptides encoded by them and to their use as targets for fungicides and to their use for identifying novel, fungicidally active compounds, and to methods of finding modulators of these polypeptides and, finally, to transgenic organisms containing sequences encoding fungal polypeptides with the function of a fructose-1,6-bisphosphate aldolase.

The invention relates to nucleic acids which encode fungal polypeptides with the biological activity of fructose-1,6-bisphosphate aldolases, to the polypeptides encoded by them and to their use as targets for fungicides and to their use for identifying novel, fungicidally active compounds, and to methods of finding modulators of these polypeptides and, finally, to transgenic organisms containing sequences encoding fungal polypeptides with the function of a fructose-1,6-bis-phosphate aldolase.

Undesired fungal growth which leads every year to considerable damage, for example in agriculture, can be controlled by the use of fungicides. The demands made on fungicides have increased constantly with regard to their activity, their costs and above all their ecological soundness. There exists therefore a demand for novel substances or classes of substances which can be developed into potent and ecologically sound novel fungicides. In general, it is customary to search for such novel lead structures in greenhouse tests. However, such tests require a high input of labour and a high financial input. The number of the substances which can be tested in the greenhouse is, accordingly, limited. An alternative to such tests is the use of what are known as high-throughput screening methods (HTS). This involves testing a large number of individual substances with regard to their effect on individual cells, individual gene products or genes in an automated method. When certain substances are found to have an effect, they can be studied in conventional screening methods and, if appropriate, developed further.

Advantageous targets for fungicides are frequently searched for in essential biosynthesis pathways. Ideal fungicides are, moreover, those substances which inhibit gene products which have a decisive importance in the manifestation of the pathogenicity for a fungus. An example of such a fungicide is, for example, the active substance carpropamid, which inhibits fungal melanin biosynthesis and thus prevents the formation of intact appressoria (adhesion organs). However, there is only a very small number of known gene products which play such a role for fungi. Moreover, fungicides are known which lead to auxotrophism of the target cells by inhibiting corresponding biosynthesis pathways and, as a consequence, to the loss of pathogenicity. Thus, for example, the inhibition of adenosin deaminase upon addition of ethirimol leads to a significantly reduced pathogenicity in Blumeria graminis.

Fructose-1,6-bisphosphate aldolase (EC 4.1.2.13), also known under the name fructose-1,6-bisphosphate triosephosphate-lyase, termed aldolase hereinbelow, catalyzes the aldol cleavage of fructose-1,6-bisphosphate (1) into glycerinaldehyde-3-phosphate (2) and dihydroxyacetone phosphate (3).

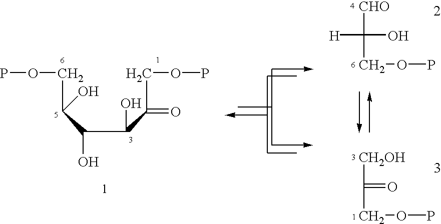

This reaction is a central step in glycolysis and gluconeogenesis.

Two classes of aldolases can be distinguished on the basis of their reaction mechanism (Marsh and Lebherz, 1992).

Class I aldolases are found in Archaebacteria, Eubacteria (for example Micrococcus aerogenes), higher plants, mammals and protozoans. The enzyme consists of a homotetramer, with each subunit approximately 40 kDa in size. During aldol cleavage, a Schiff base with the ε-amino group of a lysine residue in the active pocket of the enzyme is formed.

Class II aldolases are found in Archaebacteria and Eubacteria and also in fungi. Being homodimers, they consist of two subunits approximately 40 kDa in size. They have a characteristic Zn²⁺⁻ ion in the active centre.

Aldolase genes have been cloned from a variety of organisms, for example from E. coli (Swissprot Accession No.: P71295), Homo sapiens (Swissprot Accession No.: P05062), Arabidopsis thaliana (Swissprot Accession No.: P22197) or Saccharomyces cerevisiae (Swissprot Accession No.: P14540).

The sequence similarity within classes I and II is significant, while the sequence identity between representatives of classes I and II is less than 20%.

Aldolase from both classes is thoroughly characterized biochemically. Crystal structures exist both of the class II and class I aldolase (Hall et al, 1999; Gamblin et al., 1990).

Despite this outstanding characterization of fructose-1,6-bisphosphate aldolase, the enzyme has never been recognized as target for fungicidal active compounds. So far, the utilization of this interesting enzyme is restricted to clinical chemistry, where fructose-1,6-bisphosphate aldolase is used for the diagnosis of liver diseases and myocardial infarction (Willnow, 1985).

The complete cDNA sequence and the corresponding genes (genomic sequence, chromosome 1, start 65904-65790 (Exon 1) and 65571-64607 (Exon 2), 65789-65572 (Intron 1), see SEQ ID NO: 1) from Ustilago maydis encoding fructose-1,6-bisphosphate aldolase have been isolated within the scope of the present invention.

The smut fungus Ustilago maydis, a Basidiomycete, attacks maize plants. The disease occurs in all areas where maize is grown, but gains importance only during dry years. Typical symptoms are the gall-like, fist-sized swellings (blisters) which are formed on all aerial plant parts. The galls are first covered by a whitish-grey coarse membrane. When the membrane ruptures, a black mass of ustilospores, which is first greasy and later powdery, is released. Further species of the genus Ustilago are, for example, U. nuda (causes loose smut on barley and wheat), U. nigra (causes black smut of barley), U. hordei (causes covered smut of barley) and U. avenae (causes loose smut of oats).

By means of knock-out analyses in the Basidiomycete Ustilago maydis it has, surprisingly, been found within the scope of the present invention that the enzyme essential for survival of the organism is also in this phytopathogenic fungus, as is the case in the Ascomycete Saccharomyces cerevisiae (see also Schwelberger et al., 1980). This allows the conclusion that fructose-1,6-bisphosphate aldolase plays an important role, not only for a specific fungus, in this case Saccharomyces cerevisiae, but for fungi in general. Fructose-1,6-bisphosphate aldolase was thus recognized for the first time as an optimal target for the search for new, specific fungicides, precisely in phytopathogenic fungi, and it was thus made possible to identify, with the aid of this target, lead structures which may be entirely new and which inhibit fructose-1,6-bisphosphate aldolase and which can be used as fungicides.

It has further been found within the scope of the present invention that fructose-1,6-bisphosphate aldolase can be used for identifying substances in suitable test methods which affect the activity of the enzyme. In addition to a fructose-1,6-bisphosphate aldolase from a phytopathogenic fungus, which is characterized by its amino acid sequence and the nucleic acid sequence encoding it, suitable test methods for identifying modulators of the enzyme are also provided.

It has furthermore been found within the scope of the present invention that fructose-1,6-bisphosphate aldolase is indeed inhibited by active compounds and that a fungal organism treated with these active compounds can be damaged or killed by the treatment with these active compounds, that is to say that fructose-1,6-bisphosphate aldolase inhibitors can also be used as fungicides. For example, it is shown in the present invention that the inhibition of aldolase with substances identified in a test system leads to destruction of the treated fungi both in synthetic media and on the plant.

Even though it was already known that the gene encoding the Saccharomyces cerevisiae fructose-1,6-bisphosphate aldolase is an essential gene, it was previously unknown that aldolase, in fungi, can be a target protein of fungicidally active substances. Thus, a gene, or gene product, which has been recognized as essential by means of a knock-out cannot be considered as a target when there is no possibility of influencing the gene product, or expression of same, by external factors, for example by small chemical molecules. Thus, it is demonstrated for the first time in the present invention that aldolase constitutes an enzyme which is vital for fungi, in particular phytopathogenic fungi, and which is accessible to inhibitors and therefore particularly suitable for use as target protein for the search for further, improved, fungicidally active compounds.

Furthermore, the Ustilago maydis aldolase is described for the first time in the present invention. The aldolase described belongs to the above-described class of aldolases. Besides the Saccharomyces cerevisiae, Neurospora crassa and Schizosaccharomyces pombe fructose-1,6-bisphosphate aldolase, only part-sequences of other fungi with an average length of 400 to 500 base pairs have been published to date and as yet, they have only been classified as potential fructose-1,6-bisphosphate, aldolases. While the sequence encoding fructose-1,6-bisphosphate aldolase from a phytopathogenic Basidiomycete was hitherto unknown, only the part-sequence of a Basidiomycete which is pathogenic to humans had been deposited in databases. This is DNA from the phytopathogenic Ascomycetes Blumeria graminis, Cladosporium fulvum and Mycosphaerella graminicola, the fungi Coccidioides immitis (an Ascomycete) and Cryptococcus neoformans (a Basidiomycete), which are pathogenic to humans, and the nonpathogenic fungi Aspergillus oryzae (an Ascomycete) and Neurospora crassa (also an Ascomycete) and E. coli, which encodes fragments of fructose-1,6-bisphosphate aldolase (see Table 1).

TABLE 1

| | | Similar- | | |
|---|---|---|---|---|
| Organism | Acc. No | ity | Identity | Length |
| Aspergillus oryzae | Trembl:Q9HGY9 | 67 | 59 | Complete |
| Blumeria graminis 1,3 | embl:AW787978; | 77 | 73 | In part |
| | AW790281 | | | |
| Blumeria graminis 2,4 | embl:AW787979; | 72 | 63 | In part |
| | AW790282 | | | |
| Cladosperium fulvum | EMBL:BE187903 | 63 | 57 | In part |
| Coccidioides immitis | Trembl:Q9HGT1 | 75 | 68 | In part |
| Cryptococcus | embl:AA051839 | 75 | 68 | In part |
| neoformans | | | | |
| Kluyvermyces lactis | EMBL:AJ272114 | 72 | 65 | In part |
| Mycosphaerella | embl:AW180694 | 67 | 61 | In part |
| graminicola | | | | |
| Neurospora crassa | Swissprot: | 72 | 66 | Complete |
| | P53444; | | | |
| | (ESTs:EMBL: | | | |
| | AI392564; | | | |
| | AI398469; | | | |
| | AI399236; | | | |
| | AI399357; | | | |
| | AI399367; | | | |
| | BF072733; | | | |
| | BF072734;) | | | |
| Saccharomyces | Swissprot:P14540 | 73 | 65 | Complete |
| cerevisiae | | | | |
| Schizosaccharomyces | Swissprot:P36580 | 73 | 66 | Complete |
| pombe | | | | |
| E. coil | Swissprot:P11604 | 60 | 53 | Complete |

List of the organisms whose sequences encoding fructose-1,6-bisphosphate aldolase are known or from which part-sequences were postulated as encoding parts of fructose-1,6-bisphosphate aldolase. Shown are the origin of the sequence information and the similarity and identity of the sequences or sequence fragments with the Ustilago maydis fructose-1,6-bisphosphate aldolase (amino acid level). Phytopathogenic organisms are printed in bold and organisms which are pathogenic to humans are italicized.

The putative part-sequences, known as ESTs, can now be confirmed as sequences encoding fructose-1,6-bisphosphate aldolase by means of the known Saccharomyces sequence and the Ustilago maydis sequence according to the invention.

Based on the comparisons carried out within the scope of the present invention (see also Table 1), it can now be stated that the Ustilago maydis aldolases and the aldolases from other fungi such as, for example, Saccharomyces cerevisiae or Schizosaccharomyces pombe have a considerable degree of homology with each other, which is why polypeptides which are homologous to the Ustilago maydis fructose-1,6-bisphosphate aldolase and which are encoded by correspondingly homologous nucleic acids can also be used as partners for molecular interactions (targets) of fungicidal active compounds. The homologous nucleic acids or polypeptides from phytopathogenic fungi are of particular interest. Phytopathogenic Basidiomycetes can be used especially preferably for this purpose. Owing to the high degree of homology between the fructose-1,6-bisphosphate aldolases, the fructose-1,6-bisphosphate aldolases from fungi which are pathogenic to humans (see Table 1) or the nucleic acids encoding them may also be used for identifying inhibitors of the enzyme. Analogously, fructose-1,6-bisphosphate aldolase inhibitors may also display an activity against fungi which are pathogenic to humans and may be used as antimycotics.

This is why the present invention fully encompasses the use of fungal fructose-1,6-bisphosphate aldolases, in particular from Ascomycetes, Basidiomycetes and Oomycetes, very particularly from phytopathogenic fungi or phytopathogenic Basidiomycetes, and in particular from Ustilago maydis for identifying fungicidally active substances.

The abovementioned homologous polypeptides very particularly preferably take the form of those which have at least 60%, preferably 75%, particularly preferably 80%, very particularly preferably at least 95% similarity with the Ustilago maydis aldolase over a length of at least 20, preferably at least 25, particularly preferably at least 30 and very particularly preferably at least 100 consecutive amino acids and most preferably over the entire length.

Such polypeptides which are homologous to the Ustilago maydis fructose-1,6-bisphosphate aldolase, in particular to the polypeptide of SEQ ID NO: 2 and 3 and SEQ ID NO: 5 and which can be used for identifying fungal active substances need not constitute complete fungal fructose-1,6-bisphosphate aldolases, but may also only constitute fragments of these as long as they at least still have a biological activity of the complete fungal fructose-1,6-bisphosphate aldolases. Polypeptides which exert the same type of biological activity as an aldolase with an amino acid sequence as shown in SEQ ID NO: 2 and 3 or SEQ ID NO: 5 fructose-1,6-bisphosphate aldolase are still considered as being according to the invention. In this context, the polypeptides according to the invention need not be deducible from fructose-1,6-bisphosphate aldolases from Ustilago maydis or from phytopathogenic fungi, for the abovementioned reasons. Polypeptides which are considered according to the invention are, above all, also those polypeptides which correspond to aldolases for example of the following fungi, or fragments of these, and which still have their biological activity:

Plasmodiophoromycetes, Oomycetes, Chytridiomycetes, Zygomycetes, Ascomycetes, Basidiomycetes and Deuteromycetes, for example.

Pythium species such as, for example, Pythium ultimum, Phytophthora species such as, for example, Phytophthora infestans, Pseudoperonospora species such as, for example, Pseudoperonospora humuli or Pseudoperonospora cubensis, Plasmopara species such as, for example, Plasmopara viticola, Bremia species such as, for example, Bremia lactucae, Peronospora species such as, for example, Peronospora pisi or P. brassicae, Erysiphe species such as, for example, Erysiphe graminis, Sphaerotheca species such as, for example, Sphaerotheca fuliginea, Podosphaera species such as, for example, Podosphaera leucotricha, Venturia species such as, for example, Venturia inaequalis, Pyrenophora species such as, for example, Pyrenophora teres or P. graminea (conidial form: Drechslera, syn: Helminthosporium), Cochliobolus species such as, for example, Cochliobolus sativus (conidial form: Drechslera, syn: Helminthosporium), Uromyces species such as, for example, Uromyces appendiculatus, Puccinia species such as, for example, Puccinia recondita, Sclerotinia species such as, for example, Sclerotinia sclerotiorum, Tilletia species such as, for example, Tilletia caries; Ustilago species such as, for example, Ustilago nuda or Ustilago avenae, Pellicularia species such as, for example, Pellicularia sasakii, Pyricularia species such as, for example, Pyricularia oryzae, Fusarium species such as, for example, Fusarium culmorum, Botrytis species, Septoria species such as, for example, Septoria nodorum, Leptosphaeria species such as, for example, Leptosphaeria nodorum, Cercospora species such as, for example, Cercospora canescens, Alternaria species such as, for example, Alternaria brassicae or Pseudocercosporella species such as, for example, Pseudocercosporella herpotrichoides.

Others which are of particular interest are, for example, Magnaporthe grisea, Cochliobulus heterostrophus, Nectria hematococcus and Phytophthora species.

Fungicidal active compounds which are found with the aid of the aldolases according to the invention may also interact with aldolases from fungal species which are pathogenic to humans; however, the interaction with the different aldolases which appear in these fungi need not always be equally pronounced.

The present invention therefore also relates to the use of fructose-1,6-bisphosphate aldolase inhibitors for the preparation of compositions for treating diseases caused by fungi which are pathogenic to humans.

Of particular interest in this context are the following fungi which are pathogenic to humans and which may cause the symptoms stated hereinbelow:

Dermatophytes such as, for example, Trichophyton spec., Microsporum spec., Epidermophyton floccosum or Keratomyces ajelloi, which cause, for example, Athlete's foot (tinea pedis),

Yeasts such as, for example, Candida albicans, which causes soor oesophagitis and dermatitis, Candida glabrata, Candida krusei or Cryptococcus neoformans, which may cause, for example, pulmonal cryptococcosis or else torulosis,

Moulds such as, for example, Aspergillus fumigatus, A. flavus, A. niger, which cause, for example, bronchopulmonary Aspergillosis or fungal sepsis, Mucor spec., Absidia spec., or Rhizopus spec., which cause, for example, Zygomycoses (intravasal mycoses), Rhinosporidium seeberi, which causes, for example, chronic granulomatous pharyngitis and tracheitis, Madurella myzetomatis, which causes, for example, subcutaneous mycetomes, Histoplasma capsulatum, which causes, for example, reticulo endothelial cytomycosis and Darling's disease, Coccidioides immitis, which causes, for example, pulmonary coccidioidomycosis and sepsis, Paracoccidioides brasiliensis, which causes, for example, South American blastomycosis, Blastomyces dermatitidis, which causes, for example, Gilchrist's disease and North American blastomycosis, Loboa loboi, which causes, for example, keloid blastomycosis and Lobo's disease, and Sporothrix schenckii, which causes, for example, sporotrichosis (granulomatous dermal mycosis).

Fungicidal active compounds which are found with the aid of the aldolases according to the invention can therefore also interact with aldolases from a large number of other phytopathogenic fungal species; the interaction with the different aldolases which occur in these fungi need not always be equally pronounced. This explains, inter alia, the selectivity which has been observed of the substances which are active on this enzyme.

The present invention therefore relates to nucleic acids which encode complete fructose-1,6-bisphosphate aldolases from phytopathogenic fungi, with the exception of the sequence fragments from Blumeria graminis, Cladosporium fulvum and Mycosphaerella graminicola which are listed in Table 1 and which have the sequences deposited under the stated Accession Numbers.

The present invention particularly relates to nucleic acids which encode fructose-1,6-bisphosphate aldolases from Basidiomycetes, preferably from phytopathogenic Basidiomycetes, very especially preferably from the genus Ustilago.

The present invention very especially preferably relates to nucleic acids which encode Ustilago maydis fructose-1,6-bisphosphate aldolase.

The present invention especially preferably relates to Ustilago maydis nucleic acids as shown in SEQ ID NO: 1 and 4, wihich encode a polypeptide as shown in SEQ ID NO: 2 and 3 or SEQ ID NO: 5 or active fragments thereof.

What is of particular interest in this context is the amino acid fragment comprising amino acids 80 to 220, which, in turn, comprises the region 107-110, which is important for fructose-1,6-bisphosphate aldolase and is important for zinc binding and thus part of the active centre. Two what are known as prosite motifs (Hofmann et al., 1999) are also found in this region, motif (I) spanning the amino acids 99-111 and motif (II) the amino acids 171-182. What is known as a prosite motif is identified in a search based on protein sequences and functional domains using the PROSITE programme and is suitable for predicting the function of a gene product. A prosite motif is therefore typical of a particular enzyme class. Ultimately, a prosite motif constitutes the components of a consensus sequence, and distances between the participating amino acids.

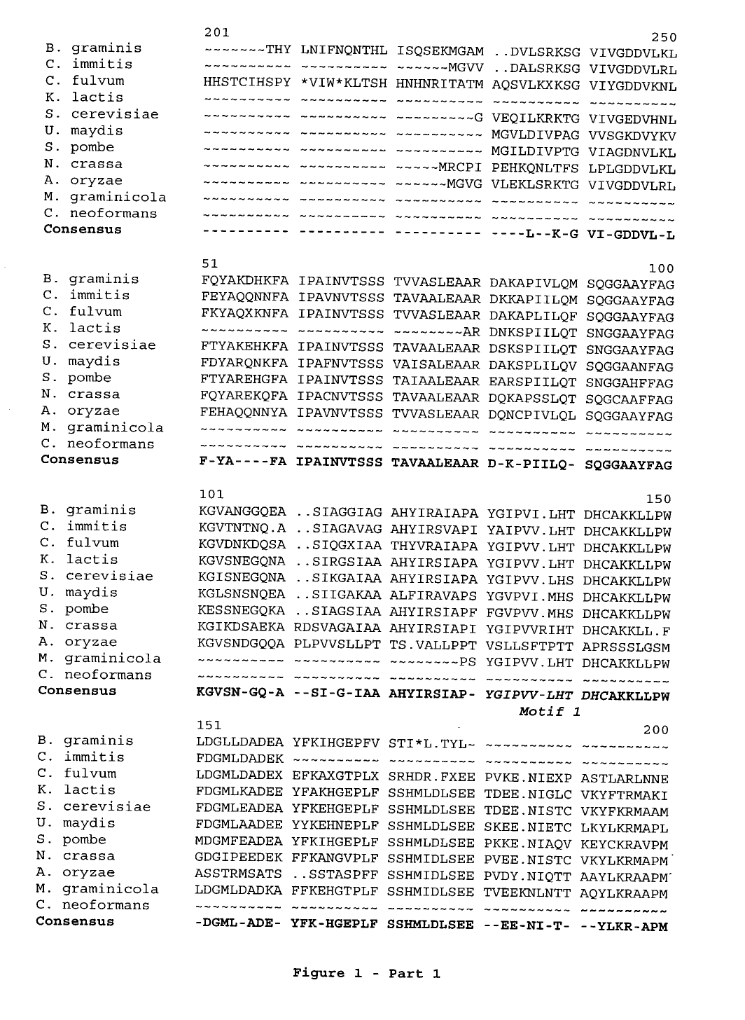

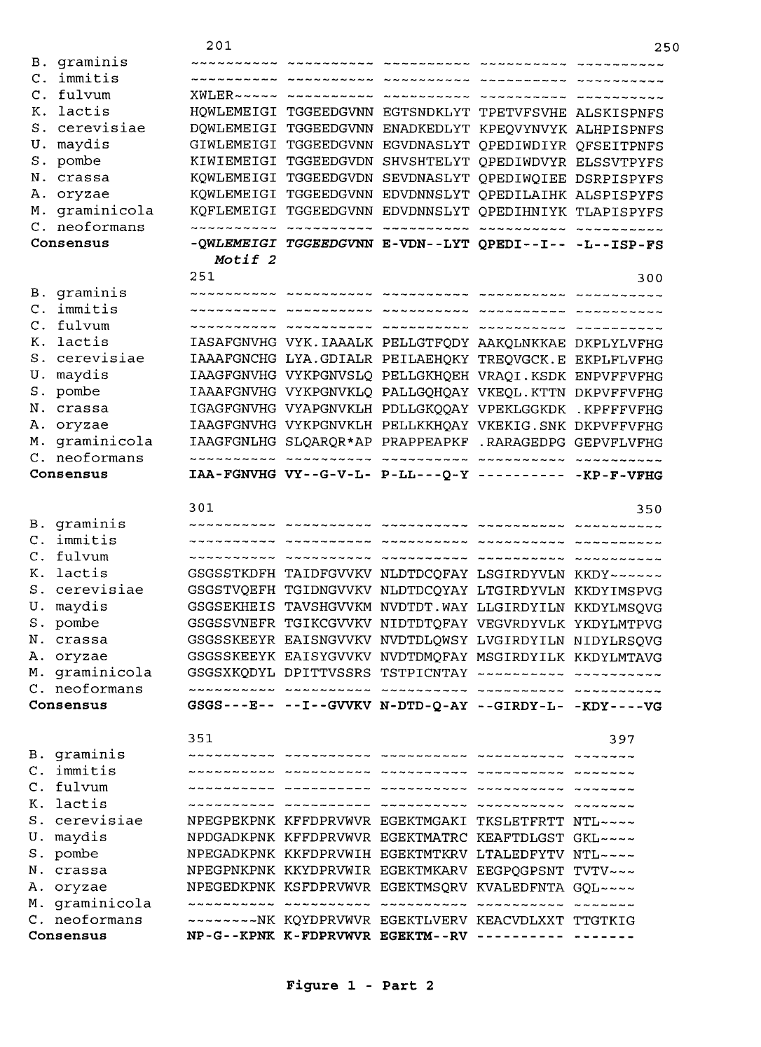

The motifs identified in a suitable search with the aid of the sequence data which is now available can be defined as follows (cf. also FIG. 1):

Prosite Motif (I): [FYVMT]-x(1,3)-[LIVMH]-[APNT]-[LIVM]-x(1,2)-[LIVM]-H-x-D-H-[GACH]

H-x-D-H is suitable for binding the catalytic Zn ion. A comparison with FIG. 1 shows that, in the present case, the consensus motif (II) can also be described specifically by

-(YGIPVV)-x-(LHTDHC)-

where x represents a position at which any amino acid is accepted or else at which there is no amino acid present.

Prosite Motif (II): [LIVM]-E-x-E-[LIVM]-G-x(2)-[GM]-[GSTA]-x-E

A comparison with FIG. 1 shows that, in the present case, the consensus motif (II) can also be described specifically by

-(LEMEIGITGGEEDGV)-

The abovementioned prosite motifs, or the specific consensus motifs, respectively, are typical of the polypeptides according to the invention, which can be defined by these consensus sequences in terms of their structure and are thereby identifiable.

Accordingly, the present invention also relates to those nucleic acids which encode polypeptides which preferably comprise both the prosite motif (1) and the prosite motif (II). The polypeptides encoded by the nucleic acids according to the invention especially preferably comprise both the consensus motif (I) and the consensus motif (II)

In particular, the nucleic acids according to the invention take the form of single-stranded or double-stranded deoxyribonucleic acids (DNA) or ribonucleic acids (RNA). Preferred embodiments are fragments of genomic DNA, which may contain introns, and cDNAs.

The nucleic acids according to the invention preferably take the form of DNA fragments which correspond to the cDNA of the nucleic acids according to the invention.

The nucleic acids according to the invention especially preferably comprise a fungal sequence selected from

a) the sequence as shown in SEQ ID NO: 1 and SEQ ID NO: 4

b) sequences encoding a polypeptide comprising the amino acid sequence as shown in SEQ ID NO: 2 and 3 and also SEQ ID NO: 5,

c) sequences encoding a polypeptide comprising the consensus motifs -(YGIPVV)-x-(LHTDHC)- and -(LEMEIGITGGEEDGV)-,

d) part-sequences of the sequences defined under a) to c) which are at least 14 base pairs in length,

e) sequences which hybridize with the sequences defined under a) to c) at a hybridization temperature of 35-52° C.,

f) sequences with at least 60%, preferably 80%, particularly preferably 90% and very particularly preferably 95% identity with the sequences defined under a) to c),

g) sequences which are complementary to the sequences defined under a) to c), and

h) sequences which, owing to the degeneracy of the genetic code, encode the same amino acid sequence as the sequences defined under a) to c).

A cDNA molecule with the sequence as shown in SEQ ID NO: 1 or SEQ ID NO: 4 encoding the Ustilago maydis fructose-1,6-bisphosphate aldolase with the SEQ ID NO: 2 and 3 or SEQ ID NO: 5 constitutes a very particularly preferred embodiment of the nucleic acids according to the invention.

The term “identity” as used in the present context refers to the number of sequence positions that are identical in an alignment. In most cases, it is indicated at a percentage of the alignment length.

The term “similarity” as used in the present context, in contrast, assumes the existence of a similarity metric, that is to say a measure for the desired assumed similarity, for example, between a valin and a threonin or a leucin.

The term “homology” as used in the present context, in turn, indicates evolutionary relationship. Two homologous proteins have developed from a shared precursor sequence. The term is not necessarily about identity or similarity, apart from the fact that homologous sequences usually have a higher degree of similarity (or occupy more identical positions in an alignment) than non-homologous sequences.

The term “complete” fructose-1,6-bisphosphate aldolase as used in the present context describes the fructose-1,6-bisphosphate aldolases encoded by the complete coding region of a transcription unit, starting with the ATG start codon and comprising all the information-bearing exon regions of the gene encoding fructose-1,6-bisphosphate aldolase which is present in the source organism, as well as the signals required for correct transcriptional termination.

The term “active fragment” as used in the present context describes nucleic acids encoding fructose-1,6-bisphosphate aldolase which are no longer complete, but still encode enzymes with the biological activity of a fructose-1,6-bisphosphate aldolase and which are capable of catalyzing a reaction characteristic of fructose-1,6-bisphosphate aldolase, as described above. Such fragments are shorter than the above-described complete nucleic acids encoding fructose-1,6-bisphosphate aldolase. In this context, nucleic acids may have been removed both up to 3′ and/or 5′ ends of the sequence, or else parts of the sequence which do not have a decisive adverse effect on the biological activity of fructose-1,6-bisphosphate aldolase may have been deleted or removed. A lower or else, if appropriate, an increased activity, which still allows the characterization or use of the resulting fructose-1,6-bisphosphate aldolase fragment, is considered as sufficient for the purposes of the term as used herein. The term “active fragment” may likewise refer to the amino acid sequence of fructose-1,6-bisphosphate aldolase; in this case, it applies analogously to what has been said above to those polypeptides which no longer contain certain portions in comparison with the above-described complete sequence, but where no decisive adverse effect is exerted on the biological activity of the enzyme. The preferred length of these fragments is 420 nucleobases, preferably 660 nucleobases, very particularly preferably 1056 nucleobases, or 140 amino acids, preferably 220 amino acids, and very especially preferably 352 amino acids, respectively.

The term “gene” as used in the present context is the name for a segment—from the genome of a cell—which is responsible for the synthesis of a polypeptide chain.

The term “to hybridize” as used in the present context describes the process in which a single-stranded nucleic acid molecule undergoes base pairing with a complementary strand. For example, starting from the sequence information which is mentioned herein or which can be deduced, DNA fragments can be isolated, in this manner, from phytopathogenic fungi other than Ustilago maydis, which fragments encode aldolases with the same or similar properties of one of the aldolases according to the invention.

Hybridization conditions are calculated approximatively by the following formula:

The melting temperature

Tm=81.5° C.+16.6{log[c(Na⁺)]}+0.41(% G+C)−(500/n)

(Lottspeich & Zorbas, 1998).

In this formula, c is the concentration and n the length of the hybridizing sequence segment in base pairs. For a sequence>100 bp, the term 500/n is dropped. The highest stringency involves washing at a temperature of 5-15° C. below Tm and an ionic strength of 15 mM Na⁺(corresponds to 0.1×SSC). If an RNA sample is used for hybridization, the melting point is 10-15° C. higher.

Preferred hybridization conditions are stated hereinbelow:

Hybridization solution: DIG Easy Hyb (Roche, Z Z) hybridization temperature: 37° C. to 50° C., preferably 42° C. (DNA-DNA), 50° C. (DNA-RNA).

| | |
|---|---|
| Wash step 1: | 2 × SSC, 0.1% SDS 2 × 5 min at room temperature; |
| Wash step 2: | 1 × SSC, 0.1% SDS 2 × 15 min at 50° C.; preferably 0.5 × |
| | SSC, 0.1% SDS 2 × 15 min at 65° C.; particularly |
| | preferably 0.2 × SSC, 2 × 15 min at 68° C. |

The degree of identity of the nucleic acids is preferably determined with the aid of the program NCBI BLASTN Version 2.0.4. (Altschul et al. 1997).

The term “heterologous promoter” as used in the present context refers to a promoter with properties other than the promoter which controls the expression of the gene in question in the original organism.

The choice of heterologous promoters depends on whether procaryotic or eucaryotic cells or cell-free systems are used for expression. Examples of heterologous promoters are the cauliflower mosaic virus 35S promoter for plant cells, the alcohol dehydrogenase promoter for yeast cells, the T3, T7 or SP6 promoters for procaryotic cells or cell-free systems, and tissue-specific promoters from phytopathogenic fungi, for example the specific promoter of the aldolase to be used in accordance with the invention.

The present invention furthermore relates to vectors containing a nucleic acid according to the invention, a regulatory region according to the invention or a DNA construct according to the invention.

Vectors which can be used are all those phages, plasmids, phagemids, phasmids, cosmids, YACs, BACs, artificial chromosomes or particles suitable for particle bombardment which are used in molecular-biological laboratories.

A preferred vector is pET15b (Novagen).

The present invention also relates to host cells containing a nucleic acid according to the invention, a DNA construct according to the invention or a vector according to the invention.

The term “host cell” as used in the present context refers to cells which do not naturally contain the nucleic acids according to the invention.

Suitable as host cells are procaryotic cells, preferably E. coli, but also eucaryotic cells such as cells of Saccharomyces cerevisiae, Pichia pastoris, phytopathogenic fungi, plants, frog oocytes and mammalian and insect cell lines.

The present invention furthermore relates to polypeptides with the biological activity of fructose-1,6-bisphosphate aldolases which are encoded by the nucleic acids according to the invention.

The polypeptides according to the invention preferably comprise an amino acid sequence selected from

a) the sequence as shown in SEQ ID NO: 2 and 3 and also SEQ ID NO: 5,

b) sequences comprising the consensus motifs -(YGIPVV)-x-(LHTDHC)- and -(LEMEIGITGGEEDGV)-,

c) part-sequences of the sequences defined under a) and b) which are at least 15 amino acids in length,

d) sequences which have at least 60%, preferably 80% and especially preferably 90% identity with the sequences defined under a) and b), and

e) sequences with the same biological activity as the sequences defined under a) and b).

The term “polypeptides” as used in the present context refers not only to short amino acid chains which are generally referred to as peptides, oligopeptides or oligomers, but also to longer amino acid chains which are normally referred to as proteins. It encompasses amino acid chains which can be modified either by natural processes, such as post-translational processing, or by chemical prior-art methods. Such modifications may occur at various sites and repeatedly in a polypeptide, such as, for example, on the peptide backbone, on the amino acid sidechain, on the amino and/or the carboxyl terminus. For example, they encompass acetylations, acylations, ADP ribosylations, amidations, covalent linkages to flavins, haem moieties, nucleotides or nucleotide derivatives, lipids or lipid derivatives or phosphatidylinositol, cyclizations, disulfide bridge formations, demethylations, cystin formations, formylations, gamma-carboxylations, glycosylations, hydroxylations, iodinations, methylations, myristylations, oxidations, proteolytic processings, phosphorylations, selenoylations and tRNA-mediated amino acid additions.

The polypeptides according to the invention may exist in the form of “mature” proteins or as part of larger proteins, for example as fusion proteins. They can furthermore exhibit secretion or leader sequences, pro-sequences, sequences which allow simple purification, such as polyhistidin residues, or additional stabilizing amino acids. The proteins according to the invention may also exist in the form in which they are naturally present in the source organism, from which they can be obtained directly, for example.

The term “complete aldolase” as used in the present context describes an aldolase which is encoded by a complete coding region of a transcription unit starting with the ATG start codon and comprising all information-bearing exon regions of the gene encoding aldolase which is present in the source organism, and signals required for correct transcriptional termination.

In comparison with the corresponding regions of naturally occurring aldolases, the polypeptides according to the invention can have deletions or amino acid substitutions, as long as they still exert at least one biological activity of the complete aldolases. Conservative substitutions are preferred. Such conservative substitutions encompass variations, one amino acid being replaced by another amino acid from among the following group:

| | |
|---|---|
| 1. | Small aliphatic residues, unpolar residues or residues of little polarity: |
| | Ala, Ser, Thr, Pro and Gly; |
| 2. | Polar, negatively charged residues and their amides: Asp, Asn, Glu |
| | and Gln; |
| 3. | Polar, positively charged residues: His, Arg and Lys; |
| 4. | Large aliphatic unpolar residues: Met, Leu, Ile, Val and Cys; and |
| 5. | Aromatic residues: Phe, Tyr and Trp. |

Preferred conservative substitutions can be seen from the following list:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

The present invention therefore also relates to the use of polypeptides which exert at least one biological activity of the fructose-1,6-bisphosphate aldolase and which comprise an amino acid sequence with at least 60%, preferably 80%, identity and very especially preferably 95% identity with the Ustilago maydis sequence as shown in SEQ ID NO: 1.

The term “biological activity of an aldolase” as used in the present context means the ability to catalyze the aldol cleavage of fructose-1,6-bisphosphate.

The nucleic acids according to the invention can be prepared in the customary manner. For example, all of the nucleic acid molecules can be synthesized chemically, or else short sections of the nucleic acids according to the invention can be synthesized chemically, and such oligonucleotides can be radiolabelled or labelled with a fluorescent dye. The labelled oligonucleotides can also be used for screening cDNA libraries generated starting from mRNA from, for example, phytopathogenic fungi. Clones with which the labelled oligonucleotides hybridize are chosen for isolating the DNA fragments in question. After characterization of the DNA which has been isolated, the nucleic acids according to the invention are obtained in a simple manner.

Alternatively, the nucleic acids according to the invention can also be generated by means of PCR methods using chemically synthesized oligonucleotides.

The term “oligonucleotide(s)” as used in the present context refers to DNA molecules composed of 10 to 50 nucleotides, preferably 15 to 30 nucleotides. They are synthesized chemically and can be used as probes.

Moreover, host cells containing the nucleic acids according to the invention may be cultured under suitable conditions in order to prepare the polypeptides according to the invention, in particular the polypeptide encoded by the nucleic acid sequence as shown in SEQ ID NO: 1. The desired polypeptides can then be isolated in the customary manner from the cells or the culture medium. Alternatively, the polypeptides may be generated in in-vitro systems.

To prepare the Ustilago maydis aldolase according to the invention, it is possible, for example, to express the gene recombinantly in Escherichia coli and to prepare an enzyme preparation from E. coli cells.

One possible aldolase purification method is based on preparative electrophoresis, FPLC, HPLC (for example using gel filtration columns, reversed-phase columns or mildly hydrophobic columns), gel filtration, differential precipitation, ion-exchange chromatography or affinity chromatography.

A rapid method of isolating the polypeptides according to the invention which are synthesized by host cells using a nucleic acid to be used in accordance with the invention starts with expressing a fusion protein, where the fusion moiety may be purified in a simple manner by affinity purification. For example, the fusion moiety may be a 6-HIS type, in which case the fusion protein can be purified on a nickel-NTA affinity column. The fusion moiety can be removed by partial proteolytic cleavage, for example at linkers between the fusion moiety and the polypeptide according to the invention which is to be purified. The linker can be designed in such a way that it includes target amino acids, such as arginin and lycin residues, which define sites for trypsin cleavage. Standard cloning methods using oligonucleotides may be employed for generating such linkers.

Other purification methods which are possible are based, in turn, on preparative electrophoresis, FPLC, HPLC (e.g. using gel filtration columns, reversed-phase columns or mildly hydrophobic columns), gel filtration, differential precipitation, ion-exchange chromatography and affinity chromatography.

The terms “isolation or purification” as used in the present context mean that the polypeptides according to the invention are separated from other proteins or other macromolecules of the cell or of the tissue. The protein content of a composition containing the polypeptides according to the invention is preferably at least 10 times, especially preferably at least 100 times, higher than in a host cell preparation.

The polypeptides according to the invention may also be affinity-purified without fusion moiety with the aid of antibodies which bind to the polypeptides.

The present invention in particular also relates to a method of finding chemical compounds which interact with aldolase and modify its properties. Owing to the important function of aldolase, modulators which affect the activity constitute novel fungicidal active compounds. Modulators may be agonists or antagonists, or inhibitors or activators.

The present invention likewise relates to the use of the polypeptides according to the invention in methods for finding chemical compounds which bind to fructose-1,6-bisphosphate aldolase and modify its properties.

Owing to the property of acting as inhibitors of fungal aldolase, in particular of aldolase of phytopathogenic fungi, methods which inhibit fructose-1,6-bisphosphate aldolase and which are found by means of suitable methods may also be used as optionally labelled competitors in methods of finding further inhibitors of fungal aldolase which need not belong to this group of compounds.

The use of the nucleic acids or polypeptides according to the invention in a method according to the invention makes it possible to find compounds which bind to the polypeptide according to the invention. The latter can then be used as fungicides, for example in plants, or as antimycotic active compounds in humans and animals. For example, host cells which contain the nucleic acids according to the invention and which express the corresponding polypeptides, or the gene products themselves, are brought into contact with a compound or a mixture of compounds under conditions which permit the interaction of at least one compound with the host cells, the receptors or the individual polypeptides.

In particular, the present invention relates to a method which is suitable for identifying fungal active compounds which interact with fungal polypeptides with the biological activity of a fructose-1,6-bisphosphate aldolase, preferably with fructose-1,6-bisphosphate aldolase from phytopathogenic fungi, especially preferably with fructose-1,6-bisphosphate aldolase from Ustilago, and polypeptides which are homologous thereto and which have the abovementioned consensus sequence. However, the methods can also be carried out with a polypeptide which is homologous to fructose-1,6-bisphosphate aldolase and which is derived from a species other than those mentioned herein. Methods which use other fructose-1,6-bisphosphate aldolase than the one according to the invention are part of the present invention.

A large number of assay systems for the purpose of assaying compounds and natural extracts are designed for high throughput numbers in order to maximize the number of substances assayed within a given period. Assay systems based on cell-free processes require purified or semi-purified protein. They are suitable for an “initial” assay, which aims mainly at detecting any possible effect of a substance on the target protein.

To find modulators, a synthetic reaction mix (for example in-vitro transcription products) or a cellular component such as a membrane, a compartment or any other preparation containing the polypeptides according to the invention are incubated together with a labelled substrate or ligand of the polypeptides in the presence and absence of a candidate compound or a candidate molecule which can be an agonist or antagonist. The ability of the candidate molecule to increase or to inhibit the activity of the polypeptides according to the invention can be identified on the basis of increased or reduced binding of the labelled ligand or increased or reduced conversion of the labelled substrate. Molecules which bind well and which lead to an increased activity of the polypeptides according to the invention are agonists. Molecules which bind well and which inhibit the biological activity of the polypeptides according to the invention are good antagonists. They may also take the form of inhibitors of the abovementioned class of fungicidal substances, but entirely new classes of substances too may show this modulatory activity. What is of particular interest is the identification of aldolase inhibitors, which can be achieved by the method described. In this case, a candidate compound can be identified in an assay described hereinabove and hereinbelow by way of inhibition of the biological activity of aldolase (inhibition assay).

Detection of the biological activity of the polypeptides according to the invention can be improved by what is known as a reporter system. In this aspect, reporter systems comprise, but are not restricted to, colorimetric or fluorimetric substrates which are converted into a product, or a reporter gene which responds to changes in the activity or the expression of the polypeptides according to the invention, or other known binding assays.

A further example of a method by which modulators of the polypeptides according to the invention can be found is a displacement assay in which the polypeptides according to the invention and the potential modulator are combined, under suitable conditions, and a molecule which is known to bind to the polypeptides according to the invention, such as a natural substrate or ligands or a substrate or ligand mimetic. The polypeptides according to the invention can themselves be labelled, for example fluorimetrically or colorimetrically, so that the number of the polypeptides which are bound to a ligand or which have undergone a conversion can be determined accurately. The efficacy of an agonist or antagonist can be determined in this manner.

Effects such as cell toxicity are, as a rule, ignored in these in-vitro systems. The assay systems check not only inhibiting, or suppressive effects of the substances, but also stimulatory effects. The efficacy of a substance can be checked by concentration-dependent assay series. Control mixtures without test substances can be used for assessing the effects; here, the candidate substance is being assayed in vivo for its damaging or destroying effect on one or more fungi.

Owing to the host cells containing nucleic acids encoding fructose-1,6-bisphosphate aldolase and available with reference to the present invention, but also owing to the corresponding, homologous fructose-1,6-bisphosphate aldolases from other species which can be identified by reference to the present invention, the development of a cell-based assay system for identifying substances which modulate the activity of the polypeptides according to the invention, is made possible.

Thus, yet another possibility of identifying substances which modulate the activity of the polypeptides according to the invention is what is known as the scintillation proximity assay (SPA), see EP 015 473. This assay system exploits the interaction of a polypeptide (for example U. maydis fructose-1,6-bisphosphase aldolase) with a radiolabelled ligand (for example a small organic molecule or a second radiolabelled protein molecule). Here, the polypeptide is bound to microspheres or beads which are provided with scintillating molecules. As the radioactivity declines, the scintillating substance in the microsphere is excited by the subatomic particles of the radiolabel, and a detectable photon is emitted. The assay conditions are optimized so that only those particles emitted from the ligand lead to a signal which are emitted by a ligand bound to the polypeptide according to the invention.

In one possible embodiment, the U. maydis fructose-1,6-bisphosphate aldolase is bound to the beads, either together with, or without, interacting or binding test substances. Test substances which can be used are, inter alia, fragments of the polypeptide according to the invention. For example, a radiolabelled ligand might be a labelled, non-cleavable fructose-1,6-bisphosphate analog. When a binding ligand binds to the immobilized fructose-1,6-bisphosphate aldolase, this ligand should inhibit or nullify an existing interaction between the immobilized fructose-1,6-bisphosphate aldolase and the labelled ligand in order to bind itself in the zone of the contact area. Once binding to the immobilized fructose-1,6-bisphosphate aldolase has taken place, it can be detected with reference to a flash of light. Accordingly, an existing complex between an immobilized and a free, labelled ligand is destroyed by the binding of a test substance, which leads to a decline in the intensity of the light flash detected. In this case, the assay system takes the form of a complementary inhibition system.

The term “competitor” as used in the present context refers to the property of the compounds to compete with other, possibly yet to be identified, compounds for binding to aldolase and to displace the latter, or being displaced by the latter, from the enzyme.

The term “agonist” as used in the present context refers to a molecule which accelerates or increases the aldolase activity.

The term “antagonist” as used in the present context refers to a molecule which slows down or prevents the aldolase activity.

The term “modulator” as used in the present context is the generic term for agonist or antagonist. Modulators can be small organochemical molecules, peptides or antibodies which bind to the polypeptides according to the invention. Moreover, modulators can be small organochemical molecules, peptides or antibodies which bind to a molecule which, in turn, binds to the polypeptides according to the invention, thus influencing their biological activity. Modulators can be natural substrates and ligands, or structural or functional mimetics of these.

The term “fungicide” or “fungicidal” as used in the present context is the generic term for substances for controlling phytopathogenic fungi and for substances for controlling fungi which are pathogenic for humans or animals. Thus, the term also extends to substances which can be used as antimycotics. In a preferred meaning, the term relates to substances for controlling phytopathogenic fungi.

The modulators are preferably small organochemical compounds.

Binding of the modulators to aldolase can modify the cellular processes in a manner which leads to the destruction of the phytopathogenic fungi treated therewith.

The present invention therefore also relates to modulators of fungal fructose-1,6-bisphosphate aldolases, preferably of fructose-1,6-bisphosphate aldolases from phytopathogenic fungi, which are found with the aid of a method of identifying aldolase modulators, which method is described in the present application.

The present invention furthermore comprises methods of finding chemical compounds which modify the expression of the polypeptides according to the invention. Such “expression modulators”, too, may be new fungicidal active compounds. Expression modulators can be small organochemical molecules, peptides or antibodies which bind to the regulatory regions of the nucleic acids encoding the polypeptides according to the invention. Moreover, expression modulators may be small, organochemical molecules, peptides or antibodies which bind to a molecule which, in turn, binds to regulatory regions of the nucleic acids encoding the polypeptides according to the invention, thus influencing their expression. Expression modulators may also be antisense molecules.

The present invention likewise relates to the use of modulators of the polypeptides according to the invention or of the expression modulators as fungicides.

The present invention likewise relates to expression modulators of fructose-1,6-bisphosphate aldolases which are found with the aid of the above-described method of finding expression modulators.

The methods according to the invention include high-throughput screening (HTS) and ultra-high-throughput screening (UHTS). Both host cells and cell-free preparations which comprise the nucleic acids and/or the polypeptides according to the invention may be used.

The invention furthermore relates to antibodies which bind specifically to the polypeptides according to the invention or fragments of these. Such antibodies are raised in the customary manner. For example, said antibodies may be produced by injecting a substantially immunocompetent host with a certain amount of a polypeptide according to the invention or a fragment thereof which is effective for antibody production, and subsequently obtaining this antibody. Furthermore, an immortalized cell line which produces monoclonal antibodies may be obtained in a manner known per se. The antibodies may be labelled with a detection reagent, if appropriate. Preferred examples of such a detection reagent are enzymes, radiolabelled elements, fluorescent chemicals or biotin. Instead of the complete antibody, fragments may also be employed which have the desired specific binding properties.

The nucleic acids according to the invention can likewise be used for generating transgenic organisms such as bacteria, plants or viruses, preferably for generating transgenic plants and fungi, especially preferably for generating transgenic fungi. These can be employed for example in assay systems which are based on an expression, of the polypeptides according to the invention or their variants, which deviate from the wild type. They furthermore include any transgenic plants or fungi in which the expression of the polypeptides according to the invention or variants of these is altered by modifying genes other than those described hereinabove or by modifying gene control sequences (for example promoters). The transgenic organisms are also of interest for (over)producing the polypeptide according to the invention; here, for example, fungi (for example yeast or Ustilago maydis) which show a higher degree of expression of the polypeptide according to the invention in comparison with their natural form are particularly suitable for use in methods (indeed also HTS methods) for identifying modulators of the polypeptide.

The most developed vector system for generating transgenic plants is a plasmid from the bacterium Agrobacterium tumefaciens. In nature, A. tumefaciens infects plants and generates tumours termed crown galls. These tumours are caused by the Ti plasmid (tumour-inducing) of A. tumefaciens. The Ti plasmid incorporates part of its DNA, termed T-DNA, into the chromosomal DNA of the host plant. A means of removing the tumour-inducing regions from the DNA of the plasmid, but retaining its property of introducing genetic material into the plants, has been developed. Then, a foreign gene, for example one of the nucleic acids according to the invention or a construct according to the invention, can be incorporated into the Ti plasmid with the aid of customary recombinant DNA techniques. The recombinant plasmid is then reinserted into A. tumefaciens, which can be then used for infecting a plant cell culture. However, the plasmid can also be inserted directly into the plants, where it incorporates itself into the chromosomes. Regeneration of such cells into intact organisms gives rise to plants containing the foreign gene and also expressing it, i.e. producing the desired gene product.

While A. tumefaciens infects dicotyledonous plants with ease, it is of limited use as vector for the transformation of monocotyledonous plants, which include a large number of agriculturally important crop plants such as maize, wheat or rice, since it does not infect these plants readily. Other techniques, for example “DNA guns”, what is known as the particle gun method, are available for the transformation of such plants. In this method, minute titanium or gold microspheres are fired into recipient cells or tissue, either by means of a gas discharge or by a powder explosion. The microspheres are coated with DNA of the genes of interest, whereby the latter reach the cells and are gradually detached and incorporated into the genome of the host cells.

Only a few of the cells which are exposed to the foreign hereditary material are capable of integrating it stably into their homologous hereditary material. In a tissue which is used for gene transfer, the nontransgenic cells predominate. During the regeneration into the intact plant, it is therefore necessary to apply a selection which provides an advantage for the transgenic cells. In practice, marker genes which are transferred into the plant cells are used for this purpose. The products of these genes inactivate an inhibitor, for example an antibiotic or herbicide, and thus allow the transgenic cells to grow on the nutrient medium supplemented with the inhibitor. However, genes which encode an enzyme which can then be detected are less problematic. These also include the polypeptide according to the invention, whose enzymatic activity can be detected as described in Example 2.

In the case of the transformation with A. tumefaciens, protoplasts (isolated cells without cell wall which, in culture, take up foreign DNA in the presence of certain chemicals or else when using electroporation) may be used instead of leaf segments.

They are kept in tissue culture until a new cell wall has formed (for example approximately 2 days in the case of tobacco). Then, agrobacteria are added, and the tissue culture is continued. A simple method for the transient transformation of protoplasts with a DNA construct is the incubation in the presence of polyethylene glycol (PEG 4000).

DNA may also be introduced into cells by means of electroporation. This is a physical method for increasing the DNA uptake into live cells. Electrical pulses temporarily increase the permeability of a biomembrane without destroying the membrane.

DNA may also be introduced by microinjection. DNA is injected into the vicinity of the nucleus of a cell with the aid of glass capillaries. However, this is difficult in the case of plant cells, which have a rigid cell wall and a large vacuole.

A further possibility is to exploit ultrasound: when cells are sonicated with soundwaves above the frequency range of hearing in humans (above 20 kHz), a temporary permeability of the membranes is also observed. When carrying out this method, the amplitude of the soundwaves must be adjusted very precisely since, otherwise, the sonicated cells burst and are destroyed.

Transgenic fungi can be generated in the manner known per se to the skilled worker (see also Examples).

The invention thus also relates to transgenic plants or fungi which contain at least one of the nucleic acids according to the invention, preferably transgenic plants such as Arabidopsis species or transgenic fungi such as yeast species or Ustilago species, and their transgenic progeny. They also encompass the plant parts, protoplasts, plant tissues or plant propagation materials of the transgenic plants, or the individual cells, fungal tissue, fruiting bodies, mycelia and spores of the transgenic fungi which contain the nucleic acids according to the invention. Preferably, the transgenic plants or fungi contain the polypeptides according to the invention in a form which deviates from the wild type. However, those transgenic plants or fungi which are naturally characterized by only a very low degree of expression, or none at all, of the polypeptide according to the invention are also considered as being according to the invention.

Accordingly, the present invention likewise relates to transgenic plants and fungi in which modifications in the sequence encoding polypeptides with the activity of a fructose-1,6-bisphosphate aldolase have been generated and which have then been selected for the suitability for generating a polypeptide according to the invention and/or an increase or reduction, obtained by mutagenesis, in the biological activity or the amount of the polypeptide according to the invention which is present in the plants or fungi.

The term “mutagenesis” as used in the present context refers to a method of increasing the spontaneous mutation rate and thus of isolating mutants. In this context, mutants can be generated in vivo with the aid of mutagens, for example with chemical compounds or physical factors which are suitable for triggering mutations (for example base analogues, UV rays and the like). The desired mutants can be obtained by selecting towards a particular phenotype. The position of the mutations on the chromosomes can be determined in relation to other, known mutations by complementation and recombination analyses. Alternatively, mutations can also be introduced into chromosomal or extrachromosomal DNA in a directed fashion (in-vitro mutagenesis, site-directed mutagenesis, error-prone PCR and the like).

The term “mutant” as used in the present context refers to an organism which bears a modified (mutated) gene. A mutant is defined by comparison with the wild type which bears the unmodified gene.

The examples which follow now demonstrate that, surprisingly, aldolase is an essential enzyme in phytopathogenic fungi and furthermore that the enzyme is a suitable target protein for identifying fungicides, that it can be used in methods of identifying fungicidally active compounds, and that the aldolase modulators which have been identified in suitable methods can be used as fungicides.

Furthermore, obtaining this enzyme from Ustilago maydis is described by way of example and, finally, the application of the present invention in the search for fungicidally active compounds is demonstrated.

The examples which follow are not limited to Ustilago maydis. Analogous methods and results are also obtained in connection with other phytopathogenic fungi.

### EXAMPLES

### Example 1

Functional Expression of the Ustilago maydis Fructose-1,6-Bisphosphate Aldolase in E. coli

The coding sequence of the Ustilago maydis fructose-1,6-bisphosphate aldolase was cloned into the expression vector pET 15b (Novagen) and transformed into the bacterial strain BL21 (DE3) (Novagen).

A preculture of fresh transformants was inoculated and incubated overnight in LB-amp (80 μg/ml) at 37° C. with shaking. The next morning, the main culture was inoculated with the preculture 1:25 in LB-amp and grown at 37° C. with shaking to OD=0.7. Then, expression of the polypeptide was induced by adding 2 mM IPTG (final concentration) and expression was carried out for 48 hours at 15° C. The cells were obtained by spinning for 10 minutes at 4° C. and 8 000 rpm in a Beckman JA14 rotor by discarding the supernatant and freezing the cell pellet in liquid nitrogen and storing it at −80° C.

A pellet from 50 ml of culture was taken up in 4 ml of break buffer (100 mM Tris-HCl, pH 8.25; 4 mM 2-mercaptoethanol; 0.3% (v/v) Protease Inhibitor for His-tagged Proteins (Sigma); 20% (v/v) glycerol; 0.1 mM ZnCl₂) and disrupted by sonication (Branson Sonifier, Output 7, 30%, 5 min) on ice. Cell debris were removed by spinning for 10 minutes at 14 000 rpm and 4° C. in a 2 ml Eppendorf vessel in an Eppendorf centrifuge. The supernatant, termed aldolase enzyme preparation hereinbelow, which contains the active protein, was divided into aliquots and stored at −20° C.

### Example 2

Enzyme Assay for Finding Fructose-1,6-Bisphosphate Aldolase Modulators

The substances to be assayed were introduced into a 384 microtiter plate in 5 μl of buffer with 10 (v/v)% dimethyl sulphoxide. The concentration of the substances was such that the final concentration of the substances in the assay which was carried out amounted to 10 μM. 25 μl of substrate and auxiliary enzyme solution (cooled to 4° C.) are pipetted in. This contains 400 mM Tris-HCl, pH 8.25; 400 mM KOAc; 1.5 μl/ml of each glycerol-3-phosphate dehydrogenase/triose-phosphate isomerase and lactate dehydrogenase (auxiliary enzymes, all from Roche Biochemicals) and 6 mM of fructose-1,6-bisphosphate (substrate). 25 μl of a dilute aldolase enzyme preparation (see Example 1) were added to this mixture. Approximately 100-500 ng of total protein were employed herefor. The reaction was initiated by adding 45 μl of NADH (533 μM in 5 mM Tris-HCl, pH 8.25) (cooled to 4° C.). The decline in fluorescence was measured at λ=360/35 nm (excitation) and λ=465/35 nm (emission) for 30-45 minutes at room temperature (determined in a preliminary measurement), and the result of a measurement in the presence of a compound to be assayed were compared with the results of a measurement in the absence of a compound to be assayed.

The substances used in the test were present in the following final concentrations, or had the following activity:

c(Tris-HCl)=125 mM, c(KOAc)=100 mM, c(MESH)=1 mM, c(P inhibitor)=0.075%, (v/v), c(glycerol)=5% (v/v), c(ZnCl₂)=20 μM, c(F-1,6BP)=1.5 mM, c(NADH)=240 μM, act. (GDH)=0.0124 U, act. (TIM)=0.0581 U, act. (LDH)=0.103 U, m(aldolase)=100-500 ng.

| | |
|---|---|
| SEQ ID | Genomic sequence encoding the Ustilago maydis fructose-1,6- |
| NO: 1: | bisphosphate aldolase. The Ustilago maydis sequence contains |
| | an intron. The coding regions can be discerned. |
| SEQ ID | Amino acid sequence encoded by exon (I) of the genomic |
| NO: 2: | sequence shown in SEQ ID NO: 1 encoding Ustilago maydis |
| | fructose-1,6-bisphosphate aldolase. |
| SEQ ID | Amino acid sequence encoded by exon (II) of the genomic |
| NO: 3: | sequence shown in SEQ ID NO: 1 encoding Ustilago maydis |
| | fructose-1,6-bisphosphate aldolase. |
| SEQ ID | cDNA sequence encoding the Ustilago maydis fructose-1,6- |
| NO: 4: | bisphosphate aldolase. |
| SEQ ID | Amino acid sequence encoded by the cDNA sequence as |
| NO: 5: | shown in SEQ ID NO: 4 encoding the Ustilago maydis |
| | fructose-1,6-bisphosphate aldolase. |
| FIG. 1: | Multiple alignment of the sequences of Table 1 using the |
| | programme pileup with GCG from the Wisconsin package, |
| | Version 10.2, 1998-2001 (gap creation penalty: 8, gap |
| | extension penalty: 2). The consensus sequence was determined |
| | using the programme Pretty from the GCG package (minimum |
| | plurality: 5) |

### REFERENCES

Altschul, S. F., Madden, T. L., Schaffer, A. A., Zhang, J. Z.; Miller W. and Lipman, D. J. (1997). Gapped BLAST and PSI-BLAST generation of protein database search programs. Nucleic Acids Res. 25: 3389-3402.

Gamblin, S. J. (1991): Activity and Specificity of Human Aldolases J. Mol. Biol. 219:573-576.

Hall D. R. et al. (1999): The crystal structure of Escherichia coli class II fructose-1,6-bisphosphate Aldolase in complex with phosphoglycolohydroxamate reveals details of mechanism and specificity. J. Mol. Biol. 287: 383-394.

Hofmann K., Bucher P., Falquet L., Bairoch A. (1999): The PROSITE database, its status in 1999. Nucleic Acids Res. 27:215-219.

Lottspeich, F., Zorbas H. (Hrsg.). (1998). Bioanalytik. Spektrum Akademischer Verlag, Heidelberg, Berlin.

Marsh J. J. and Lebherz H G (1992): Fructose-bisphosphate aldolases: an evolutionary history. TIBS 17:110-113.

P. Willnow: “Fructose-1,6-bisphosphate Aldolase” in H. Bergmeyer: Enzymatic Analysis” (1985), pp 346-353, Verlag Chemie.

Schwelberger H. G. et al. (1989): Molecular cloning, primary structure and disruption of the structural gene of Aldolase from Saccharomyces cerevisiae. Eur. J. Biochem. 180: 301-308.

1. Method of identifying fungicides, characterized in that a candidate molecule is assayed in a fructose-1,6-bisphosphate aldolase inhibition assay and those candidate molecules which have an inhibitory effect on the activity of fructose-1,6-bisphosphate aldolase are selected.

2. Method according to claim 1, characterized in that the candidate molecule selected is subsequently tested in vivo for its fungicidal action.

3. Use of fungal fructose-1,6-bisphosphate aldolase, of nucleic acid encoding it, of DNA constructs or host cells containing these nucleic acids for finding fungicidal active compounds.

4. Use of a modulator of a polypeptide with the biological activity of a fructose-1,6-bisphosphate aldolase as fungicide and/or antimycotic.

5. Modulators of the fungal fructose-1,6-bisphosphate aldolase which are identified by a method according to claim 1 or 2.

6. Nucleic acid encoding a polypeptide with the activity of a fructose-1,6-bisphosphate aldolase from phytopathogenic fungi.

7. Nucleic acid according to claim 6, characterized in that it encodes a fructose-1,6-bisphosphate aldolase from Basidiomycetes.

8. Nucleic acid according to claim 6 or 7, characterized in that it encodes fructose-1,6-bisphosphate aldolase from Ustilago.

9. Nucleic acids according to claim 6, 7 or 8, characterized in that they take the form of single-stranded or double-stranded DNA or RNA.

10. Nucleic acids according to one of claims 6 to 9, characterized in that they take the form of fragments of genomic DNA or the form of cDNA.

11. Nucleic acids according to one of claims 6 to 10, comprising a sequence selected from
a) the sequence as shown in SEQ ID NO: 1 and SEQ ID NO: 4
b) sequences encoding a polypeptide comprising the amino acid sequence as shown in SEQ ID NO: 2 and 3 and also SEQ ID NO: 5,
c) sequences encoding a polypeptide comprising the consensus motifs -(YGIPVV)-x-(LHTDHC)- and -(LEMEIGITGGEEDGV)-,
d) part-sequences of the sequences defined under a) to c) which are at least 14 base pairs in length,
e) sequences which hybridize with the sequences defined under a) to c) at a hybridization temperature of 35-52° C.,
f) sequences with at least 60%, preferably 80%, particularly preferably 90% and very particularly preferably 95% identity with the sequences defined under a) to c),
g) sequences which are complementary to the sequences defined under a) to c), and
h) sequences which, owing to the degeneracy of the genetic code, encode the same amino acid sequence as the sequences defined under a) to f).

12. DNA construct comprising a nucleic acid according to one of claims 6 to 11 and a heterologous promoter.

13. Vector comprising a nucleic acid according to one of claims 6 to 11, or a DNA construct according to claim 12.

14. Vector according to claim 13, characterized in that the nucleic acid is linked operably to regulatory sequences which ensure the expression of the nucleic acid in procaryotic or eucaryotic cells.

15. Host cell comprising a nucleic acid according to one of claims 6 to 11, a DNA construct according to claim 12 or a vector according to claim 13 or 14.

16. Host cell according to claim 15, characterized in that it takes the form of a procaryotic cell.

17. Host cell according to claim 16, characterized in that it takes the form of a eucaryotic cell.

18. Polypeptide with the biological activity of a fructose-1,6-bisphosphate aldolase, which polypeptide is encoded by a nucleic acid according to one of claims 6 to 11.

19. Polypeptide with the biological activity of a fructose-1,6-bisphosphate aldolase, which polypeptide comprises an amino acid sequence which has at least 60% similarity with the sequence as shown in SEQ ID NO: 2 and 3 or SEQ ID NO: 5.

20. Antibody which binds specifically to a polypeptide according to claim 18 or 19.

21. Method of generating a nucleic acid according to one of claims 6 to 11, comprising the following steps:
a) full chemical synthesis in a manner known per se, or
b) chemical synthesis of oligonucleotides, labelling the oligonucleotides, hybridizing the oligonucleotides with DNA of a genomic library or a cDNA library generated from genomic DNA or mRNA from fungal cells, selecting positive clones, and isolating the hybridizing DNA from positive clones, or
c) chemical synthesis of oligonucleotides and amplification of the target DNA by means of PCR.

22. Method of generating a polypeptide according to claim 18 or 19, which comprises
a) culturing a host cell according to one of claims 15 to 17 under conditions which ensure the expression of the nucleic acid according to one of claims 6 to 11, or
b) expressing a nucleic acid according to one of claims 6 to 11 in an in-vitro system, and
(c) obtaining the polypeptide from the cell, the culture medium or the in-vitro system.

23. Method of finding a compound which modifies the expression of polypeptides according to claim 18 or 19, which comprises the following steps:
a) bringing a host cell according to one of claims 15 to 17 in contact with a chemical compound or a mixture of chemical compounds,
b) determining the polypeptide concentration, and
c) identifying the compound which specifically influences the expression of the polypeptide.

24. Use of a nucleic acid according to one of claims 6 to 11, a DNA construct according to claim 12 or a vector according to claims 13 or 14 for generating transgenic plants and fungi.

25. Transgenic plants, plant parts, protoplasts, plant tissues or plant propagation materials, characterized in that, after introduction of a nucleic acid according to one of claims 6 to 11, a DNA construct according to claim 12 or a vector according to claim 13 or 14, the intracellular concentration of a polypeptide according to claim 18 or 19 is increased or reduced in comparison with the corresponding wild-type cells.

26. Transgenic fungi, fungal cells, fungal tissue, fruiting bodies, mycelia and spores, characterized in that, after introduction of a nucleic acid according to one of claims 6 to 11, a DNA construct according to claim 12 or a vector, according to claim 13 or 14, the intracellular concentration of a polypeptide according to claim 18 or 19 is increased or reduced in comparison with the corresponding wild-type cells.

27. Plants, plant parts, protoplasts, plant tissue or plant propagation materials, characterized in that they contain a polypeptide according to claim 18 or 19 whose biological activity or expression pattern is modified in comparison with the corresponding endogenous polypeptides.

28. Method of generating plants, plant parts, protoplasts, plant tissues or plant propagation materials according to claim 27, characterized in that a nucleic acid according to one of claims 6 to 11 is modified by endogenous mutagenesis.

29. Fungi, fungal cells, fungal tissue, fruiting bodies, mycelia and spores, characterized in that they contain a polypeptide according to claim 18 or 19 whose biological activity or expression pattern is modified in comparison with the corresponding endogenous polypeptides.

30. Method of generating fungi, fungal cells, fungal tissue, mycelia and spores according to claim 29, characterized in that a nucleic acid according to one of claims 6 to 11 is modified by endogenous mutagenesis.


SEQUENCE LISTING (5 sequences)

SEQ ID NO: 1 - DNA, 2002 bp, Ustilago maydis
  CDS (97)..(207)
atggtacgctgcaacaattctggagcacagcaaagctgacatgtaggccttttccctttccttttctttgatcttttttggtctccttgcctcagaatgggtgttcttgacattgttcccgccggtgttgtctccggaaaggacgtttacaaggtcttcgactacgctcgtcagaacaagtttgccatccccgccttcaacgttactgtaagtaccgcctttggctcaaggaaatggtgtctcgggacgcggactgccagcttctcgcgagctgctttctgtgataaaaaccttttcagattcaatgacaaaagtcgcataaattcgccctgttggatgattgctgacgacctcatcgcattctctcgcatttcaactacttcatggataccgctggcgaatctgccgtttttgcgatttctgaaatagtcgtcctcggtcgccatctcggctctcgaggctgctcgtgatgccaagtcgcccctgatccttcaggtctcacagggaggtgccgccaactttgccggcaagggtctctctaactcgaaccaggaggcctcgatcatcggtgccaaggccgccgctctcttcattcgcgccgttgctccctcgtacggtgtccctgttatcatgcactcggaccactgcgccaagaagctgcttccttggtttgatggcatgctcgccgccgacgaggagtactacaaggagcacaacgagccgctcttttcgtcgcacatgctcgacttatccgaggagtccaaggaggagaacatcgagacctgcctcaagtacctcaagcgcatggctcctctcggcatctggctcgagatggagatcggcattactggtggtgaagaggacggtgttaacaacgagggtgttgacaacgcctcgctctacacccagcccgaggacatctgggacatctaccgccagttctccgagattacccccaacttctccatcgccgccggtttcggtaacgttcacggtgtctacaagcccggaaacgtctcgctgcagcccgagctgctcggcaagcaccaggagcacgtccgcgctcagatcaagagcgacaaggagaacccggtcttcttcgtcttccacggcggatccggttccgagaagcacgagatcagcaccgccgtcagccacggtgtggtcaagatgaacgtcgacaccgacacgcagtgggcctacctgctcggtatccgcgactacatcttgaacaagaaggactacctcatgagccaggtcggcaaccccgatggcgccgacaagcctaacaagaagttcttcgaccctcgtgtttgggttcgcgagggtgagaagaccatggctacgcgttgcaaagaggcctttactgaccttggctccactggcaagctctaagctgttcacttgacagaagccattgactatgttttcgaaggatggcacgtacacgtacacacacatgcaaacacatgcaaacacatgcaaaatttgataatcttagcccgatctgctttgccaatacggcgcgtgccggtgcattgcgtggagtagacagatgtgaggttcaggaacgtggagcttagcaggcgtattctgggcagacgacgacgagtgggtaagcttgagatgcaaaggtcatggatctggagaccagaaggtgcatctgcccgatgcgtttcctgttaccagccttgcgggcagcacggggtgcatacacgtaacagcggggacggcggtgaccaggttctcgtcccacaagcttctgaggagcgtgccgtcggcagcgtacacttcggctcttcggctcatgctgccgatggtaaagtgaggttcgagcaaagcgttttggttccatttggcgcggaagagggtgagccatttccctgtttggttgttgtgtgggatgcgcgtcgggttggcgagaacgtcgtctgggcgcttcgagagggatgggctaggtgcgtcctgttcttgcttgatggcttcagcgacgtcttttttc

SEQ ID NO: 2 - protein, 37 aa, Ustilago maydis
MGVLDIVPAGVVSGKDVYKVFDYARQNKFAIPAFNVT

SEQ ID NO: 3 - protein, 321 aa, Ustilago maydis
SSSVAISALEAARDAKSPLILQVSQGGAANFAGKGLSNSNQEASIIGAKAAALFIRAVAPSYGVPVIMHSDHCAKKLLPWFDGMLAADEEYYKEHNEPLFSSHMLDLSEESKEENIETCLKYLKRMAPLGIWLEMEIGITGGEEDGVNNEGVDNASLYTQPEDIWDIYRQFSEITPNFSIAAGFGNVHGVYKPGNVSLQPELLGKHQEHVRAQIKSDKENPVFFVFHGGSGSEKHEISTAVSHGVVKMNVDTDTQWAYLLGIRDYILNKKDYLMSQVGNPDGADKPNKKFFDPRVWVREGEKTMATRCKEAFTDLGSTGKL

SEQ ID NO: 4 - DNA, 1077 bp, Ustilago maydis
  CDS (1)..(1074)
atgggtgttcttgacattgttcccgccggtgttgtctccggaaaggacgtttacaaggtcttcgactacgctcgtcagaacaagtttgccatccccgccttcaacgttacttcgtcctcggtcgccatctcggctctcgaggctgctcgtgatgccaagtcgcccctgatccttcaggtctcacagggaggtgccgccaactttgccggcaagggtctctctaactcgaaccaggaggcctcgatcatcggtgccaaggccgccgctctcttcattcgcgccgttgctccctcgtacggtgtccctgttatcatgcactcggaccactgcgccaagaagctgcttccttggtttgatggcatgctcgccgccgacgaggagtactacaaggagcacaacgagccgctcttttcgtcgcacatgctcgacttatccgaggagtccaaggaggagaacatcgagacctgcctcaagtacctcaagcgcatggctcctctcggcatctggctcgagatggagatcggcattactggtggtgaagaggacggtgttaacaacgagggtgttgacaacgcctcgctctacacccagcccgaggacatctgggacatctaccgccagttctccgagattacccccaacttctccatcgccgccggtttcggtaacgttcacggtgtctacaagcccggaaacgtctcgctgcagcccgagctgctcggcaagcaccaggagcacgtccgcgctcagatcaagagcgacaaggagaacccggtcttcttcgtcttccacggcggatccggttccgagaagcacgagatcagcaccgccgtcagccacggtgtggtcaagatgaacgtcgacaccgacacgcagtgggcctacctgctcggtatccgcgactacatcttgaacaagaaggactacctcatgagccaggtcggcaaccccgatggcgccgacaagcctaacaagaagttcttcgaccctcgtgtttgggttcgcgagggtgagaagaccatggctacgcgttgcaaagaggcctttactgaccttggctccactggcaagctctaa

SEQ ID NO: 5 - protein, 358 aa, Ustilago maydis
MGVLDIVPAGVVSGKDVYKVFDYARQNKFAIPAFNVTSSSVAISALEAARDAKSPLILQVSQGGAANFAGKGLSNSNQEASIIGAKAAALFIRAVAPSYGVPVIMHSDHCAKKLLPWFDGMLAADEEYYKEHNEPLFSSHMLDLSEESKEENIETCLKYLKRMAPLGIWLEMEIGITGGEEDGVNNEGVDNASLYTQPEDIWDIYRQFSEITPNFSIAAGFGNVHGVYKPGNVSLQPELLGKHQEHVRAQIKSDKENPVFFVFHGGSGSEKHEISTAVSHGVVKMNVDTDTQWAYLLGIRDYILNKKDYLMSQVGNPDGADKPNKKFFDPRVWVREGEKTMATRCKEAFTDLGSTGKL